(19) United States
(12) Patent Application Publication
(10) Pub. No.: US 2012/0165685 A1
(43) Pub. Date: Jun. 28, 2012

(54) PLASMAPHERESIS DONOR DISPLAY AND METHOD OF USE

(76) Inventors: Marc N. Weasler, West Bend, IN (US); Art Marshall, Buffalo Grove, IL (US); George J. Guffey, Volo, IL (US); Robert Carl Coon, Chicago, IL (US)

(21) Appl. No.: 12/976,765

(22) Filed: Dec. 22, 2010

(51) Int. Cl. G09B 23/28; A61B 5/11; A61B 5/021

(52) U.S. Cl. 600/485; 434/267; 600/595

(57) ABSTRACT

Certain examples provide a blood collection system including an operator user interface to allow an operator to configure the system for a blood collection procedure from a donor. The system also includes a donor display, separate from the operator user interface, arranged in the system to be within view of the donor who is to donate via the system. The donor display is to display information and instruction to the donor. The information and instruction include a first indicator regarding a progress of the blood collection procedure for the donor and a second indicator including a visual instruction to the donor regarding the blood collection procedure. The system includes a processor and a memory. The processor is to execute instructions stored in the memory to process input from and provide output to the operator user interface and the donor display.

### FIELD

The present invention relates generally to methods and apparatus for collecting a blood component, and more particularly, to methods and apparatus for display information and instructions to a donor regarding a blood collection procedure involving the donor.

### BACKGROUND

An apheresis instrument is used to separate blood components from whole blood. Such apheresis instruments are commercially available from various sources, including the Amicus® instrument, the ALYX™ blood processing system, and the Autopheresis-C™ plasmapheresis collection device, which are available from Fenwal Inc. of Lake Zurich, Ill. Such instruments, also known as “separators”, typically separate a selected blood component from whole blood by passing the blood of a donor through the instrument to separate one or more blood components from the whole blood. The remainder of the whole blood is then returned to the circulatory system of the donor. It is, therefore, an extracorporeal blood component collection process. Currently, displays are focused on the operator, and minimal, if any, information is provided to the donor.

### SUMMARY

Certain examples provide a blood collection system including an operator user interface to allow an operator to configure the system for a blood collection procedure from a donor. The system also includes a donor display, separate from the operator user interface, arranged in the system to be within view of the donor who is to donate via the system. The donor display is to display information and instruction to the donor. The information and instruction include a first indicator regarding a progress of the blood collection procedure for the donor and a second indicator including a visual prompt for donor interaction in the blood collection procedure, the visual prompt dynamically generated based on obtained sensor feedback indicative of donor blood flow. The system includes a processor and a memory. The processor is to execute instructions stored in the memory to process input from and provide output to the operator user interface and the donor display.

Certain examples provide a tangible computer readable medium including executable program instructions which, when executed by a processor, cause the processor to provide an operator user interface and a donor display. The operator user interface is to allow an operator to configure a blood collection system for a blood collection procedure from a donor. The donor display, separate from the operator user interface, is arranged in the blood collection system to be within view of the donor who is to donate via the blood collection system. The donor display is to display information and instruction to the donor. The information and instruction includes a first indicator regarding a progress of the blood collection procedure for the donor and a second indicator including a visual prompt for donor interaction in the blood collection procedure, the visual prompt dynamically generated based on obtained sensor feedback indicative of donor blood flow.

Certain examples provide a method for computer-assisted blood collection from a donor. The method includes processing data regarding donor blood flow and collected blood component volume from an ongoing blood collection procedure at a blood collection device. The method includes determining at least one of a desired donor hand squeeze intensity and a desired donor hand squeeze frequency based on the processed data. The method also includes generating a graphical indicator based on the at least one of the desired donor hand squeeze intensity and the desired donor hand squeeze frequency to instruct the donor regarding the at least one of donor hand squeeze intensity and donor hand squeeze frequency to facilitate the blood collection procedure. The method includes displaying the graphical indicator to the donor via a donor display arranged to be viewable by the donor during the ongoing blood collection procedure.

### BRIEF DESCRIPTION OF THE DRAWINGS

Certain example embodiments of the invention, together with features and the advantages thereof, may best be understood by reference to the following description taken in conjunction with the accompanying drawings, in which like reference numerals identify like elements in the figures, and in which:

FIG. 1 depicts an example plasmapheresis device including a graphical user interface display and a secondary donor prompt interface display.

FIG. 2 depicts an example plasmapheresis device including a graphical user interface display and a secondary donor prompt interface display.

FIG. 3 shows a closer view of an example user interface display.

FIG. 4 illustrates several views of example donor display(s).

FIG. 5 provides examples of a donor blood flow indicator.

FIG. 6 depicts a flow diagram for an example method for display of information and instructions to a donor.

FIG. 7 is a block diagram of an example processor system that can be used to implement systems, apparatus, and methods described herein.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DESCRIPTION OF CERTAIN EXAMPLES

It will be understood that the present invention may be embodied in other specific forms without departing from the spirit thereof. The present examples and embodiments, therefore, are to be considered in all respects as illustrative and not restrictive, and the invention is not to be limited to the details presented herein.

Although the following discloses example methods, apparatus, systems, and articles of manufacture including, among other components, firmware and/or software executed on hardware, it should be noted that such methods, apparatus, systems and articles of manufacture are merely illustrative and should not be considered as limiting. For example, it is contemplated that any or all of these firmware, hardware, and/or software components could be embodied exclusively in hardware, exclusively in software, exclusively in firmware, or in any combination of hardware, software, and/or firmware. Accordingly, while the following describes example methods, apparatus, systems, and/or articles of manufacture, the examples provided are not the only way(s) to implement such methods, apparatus, systems, and/or articles of manufacture.

In certain examples, an automated plasmapheresis system separates whole blood into 1) plasma for collection and 2) concentrated cellular components for reinfusion back to the donor. The example system includes an instrument, software, and a single-use disposable kit. The example system interfaces with a plasma collection container (e.g., a bag, bottle, and/or other container), saline and/or anti-coagulant solution(s), and a fistula. The example instrument is a microprocessor-controlled, electromechanical device including valving, pumping, and sensing mechanisms with appropriate safety systems and alarm functions. A collection process using the plasmapheresis device includes, for example, a single venipuncture and alternates between withdrawal of whole blood and separation of plasma and then reinfusion of the residual cellular concentrate. A donor remains connected to the system through the process, and fluids remain in the fluid path until the procedure is completed, for example. An operator touch screen and message display allow the operator to control the procedure, gather information on its status, and address error conditions as they may arise. Examples of automated plasmapheresis systems using plasma separation technology include Fenwal's Autopheresis-C® (also referred to as Auto-C) Plasmapheresis system.

In certain examples, the plasmapheresis system includes an enhanced user interface display, minimized or reduced nuisance alarms (e.g., weigh scale), improved alarm visual indication, and/or improved donor status/prompt display(s). The user display utilizes icon driven prompts and universal menus for a global language, where applicable, to reduce or minimize subsequent efforts in the creation, translation, and future implementations for various world languages.

Using the device, plasmapheresis can be performed in a source plasma collection facility to yield a viable plasma product for use in producing plasma derived pharmaceutical products. The plasma collected through the procedure is to be compliant to the appropriate regulatory requirements.

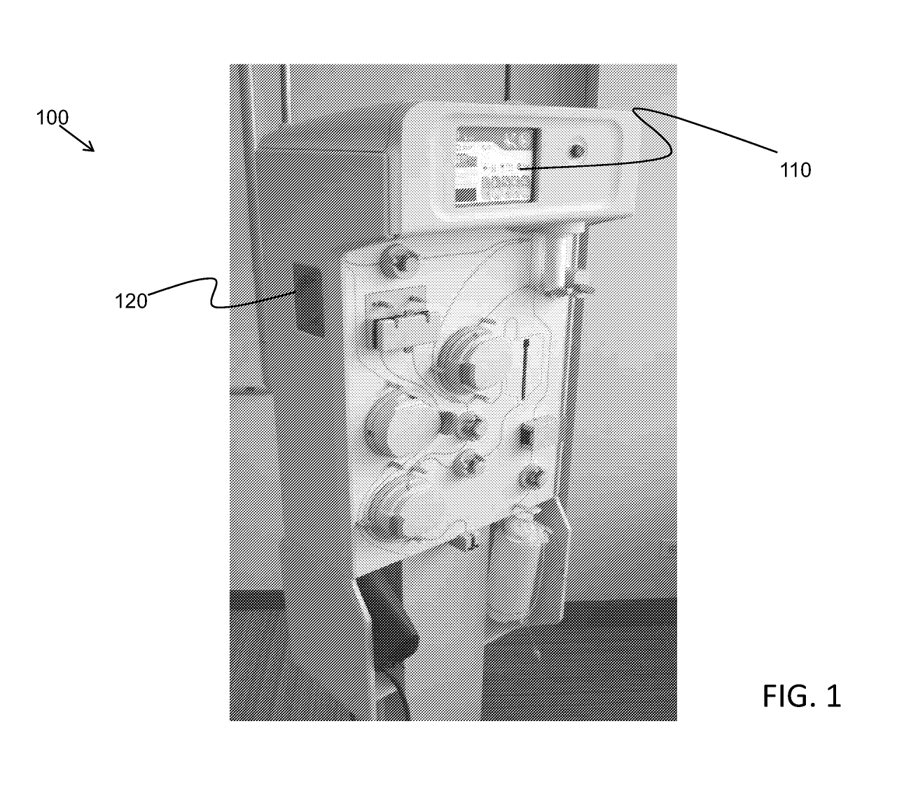

FIG. 1 depicts an example plasmapheresis device 100 including a graphical user interface display 110 and a secondary donor prompt interface display 120. Using the user interface 110, a user, such as a nurse or technician, can configure the device 100 for use in extracting blood from a donor. The interface 110 provides the user with command entry, menu buttons, collection status, donor status, etc. The interface 110 can be a touch screen interface, for example. The donor display 120 provides information, status updates, prompts, and/or other instructions to the donor connected to the device 100, for example. For example, the donor display 120 can indicate how much blood (and/or blood component) has been collected (e.g., in milliliters) compared to a target volume for collection. The donor display 120 can provide a visual indication of donor blood flow (e.g., slow, medium, fast, blue, orange, red, etc.). The visual indication of donor blood flow can graphically prompt the donor to squeeze his/her fist to increase blood flow, stop squeezing, change frequency of squeezing, change intensity of squeezing, etc. The display 120 can also include an indication of when the donation is complete (e.g., the target volume has been collected), for example.

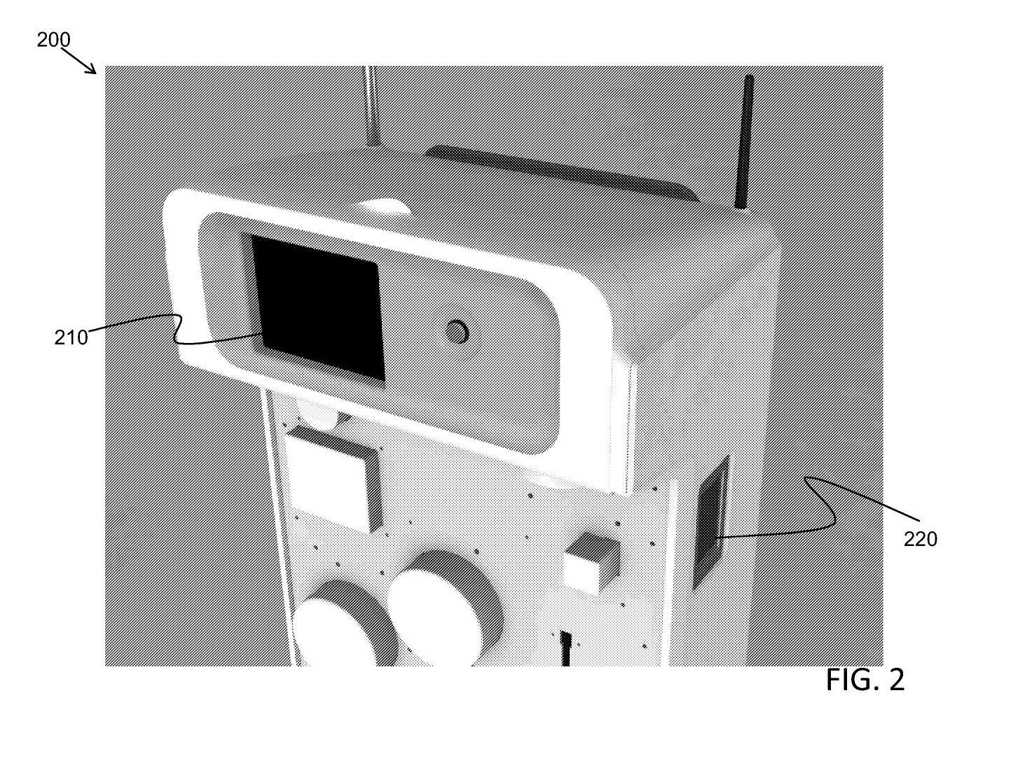

FIG. 2 illustrates another view of an example plasmapheresis device including an operator interface 210 and a donor display 220.

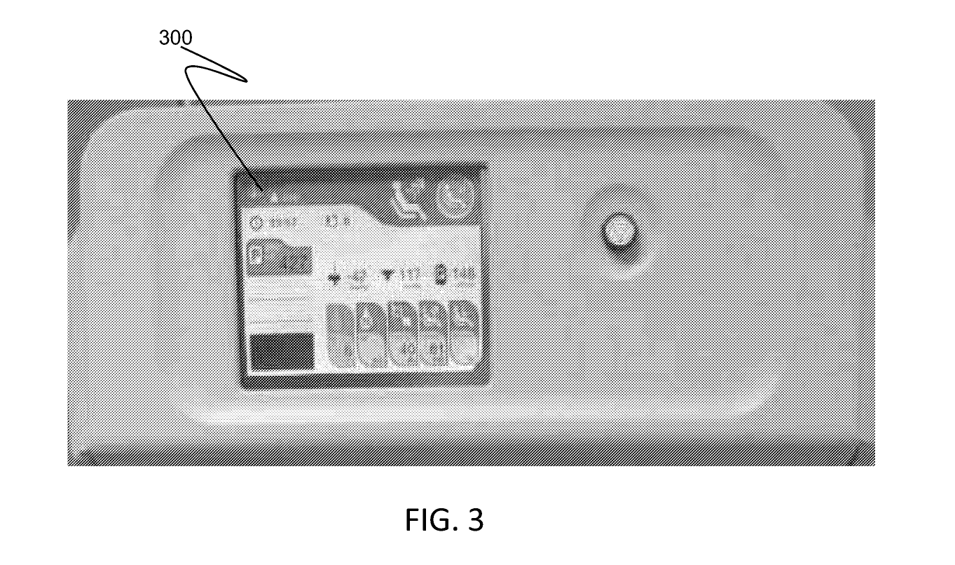

FIG. 3 shows a closer view of an example user interface display 300. The user display 300 can be used to configure a blood collection device (e.g., a plasmapheresis device), relay information regarding a donor, relay information regarding a donation, provide configuration options and/or instructions to a user, etc. The user interface 300 can be a touchscreen user interface and/or can include separate buttons, dials, knobs, and/or keys, etc., for user actuation, for example.

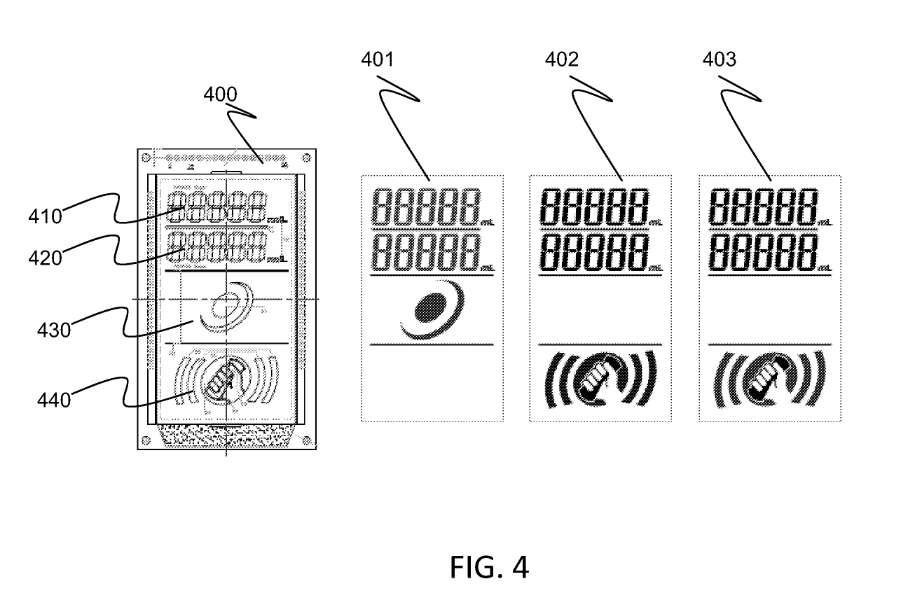

FIG. 4 illustrates several views of example donor display(s) 400-403. The donor displays 400-403 can be implemented using a liquid crystal display (LCD) and/or other display medium, for example. The donor display 400-403 can include a touchscreen and/or be a non-touchscreen display, for example. The donor display 400-403 is included in a blood collection device (e.g., a plasmapheresis device). The donor display 400-403 is arranged within/on the device to be visible to a donor connected to the device (e.g., undergoing a blood (component) collection procedure at the device). The donor display 400-403 provides information, feedback, instructions, etc., to a donor undergoing a blood (component) collection procedure at the device.

In certain examples, a bar graph and/or multi-segment (e.g., light emitting diode (LED)) display can be provided to display an indication of actual flow rate versus targeted flow rate (e.g., a percentage). The number of segments that are lit or displayed increases as the percentage of actual flow rate versus target flow rate increases (e.g., reaching the desired target flow rate). When fewer segments are lit, the more the donor is to be squeezing. As more segments are lit (approaching 100%), the donor is to squeeze less. Alternatively or in addition, as more segments are lit, the donor may be asked to squeeze more. In some examples, if a blood component is being collected from the donor and the remainder (or most of the remainder) of the blood is being returned to the donor, the donor may be instructed not to squeeze (or an instruct to squeeze may be absent) as blood is being returned to donor.

In certain examples, such as the example shown in FIG. 4, a graphical prompt is substituted for a segmented and/or percentage display. Donor collection information, such as vein and/or flow control sensing information, is used to generate one or more appropriate graphical prompts to the donor via the donor display 400-403.

The donor display 400 includes numeric sections 410, 420 to display numeric data regarding the blood collection procedure. For example, one of the numeric sections 410, 420 displays a target volume (e.g., a target plasma volume, etc.) to be collected. As the procedure transpires, the other of the numeric sections 410, 420 displays an incrementing number indicating a volume collected so far, as tracked by the device. Thus, a donor can see his/her progress based on volume collected versus target volume, for example. An indicator 430 is displayed when the procedure is complete (e.g., the targeted blood volume is collected). In certain examples, a time elapsed during the procedure is provided to the donor.

A blood flow indicator 440 is displayed to the user to indicate the donor's current blood flow rate and instruct the donor to help increase or decrease the flow rate. The blood flow indicator 440 can include a plurality of possible states to display to the donor. The states and resulting indication 440 can instruct the donor regarding a squeeze of the fist on the arm from which blood is being collected, for example. The indicator 440 can include, for example, a squeezed fist alone or in combination with one or more bars as illustrates in the examples of FIG. 4. The indicator 440 can be represented in one or more different colors (e.g., blue, red, etc.) to indicate a degree, severity, state, etc. In some examples, the indicator 440 can blink to alert a donor, instruct a donor to squeeze at a certain rate, etc. For example, the indicator 440 can alter between a fist and a fist with bar(s) if a donor response is not detected. In an example, the indicator 440 can be an animated indicator that changes color and/or number of bar(s) based on a presence and/or absence of a user response, for example. In certain examples, alphanumeric information (e.g., percentage completion, text-based updates, etc.) can be provided with the indicator 440. In certain examples, a number of fists can be displayed as the indicator 440 to convey to the donor an intensity and/or frequency to squeeze.

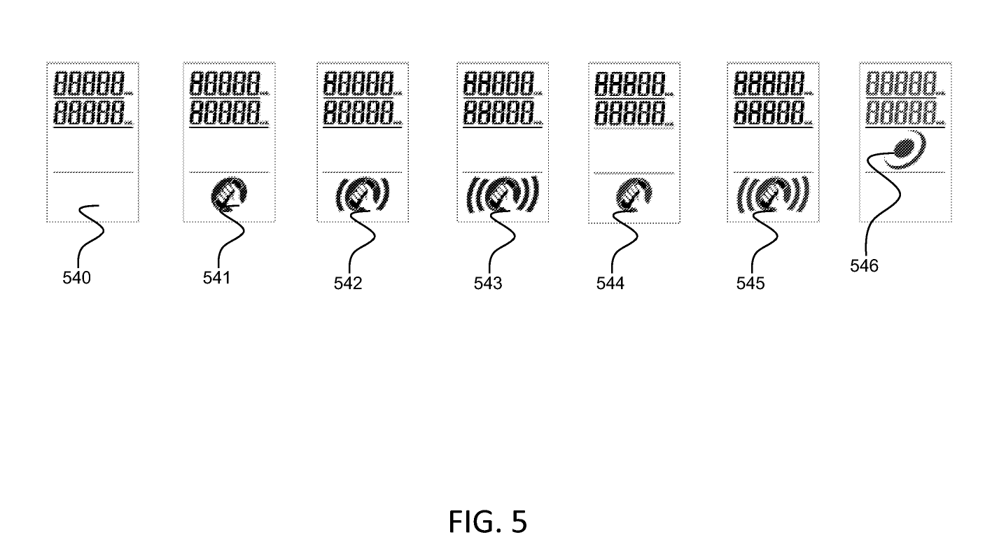

FIG. 5 provides further examples of a donor blood flow indicator 540-545. If no indicator (e.g., no fist) is shown 540, then the donor can relax, for example. A first indicator 541 (e.g., a fist) instructs the donor to lightly squeeze and/or squeeze with light frequency. A second indicator 542 (e.g., a fist with one bar) instructs the donor to squeeze normally and/or with a normal frequency. A third indicator 543 (e.g., a fist with two bars) instructs the donor to squeeze hard and/or with more frequency. In certain examples, a color of the indicator (e.g., blue or red) can change to indicate a state of blood flow and/or collection process. For example, a blue fist and/or bars 541-543 can change to a red fist and/or bars in indicators 544-545. In certain examples, the indicator can flash to instruct the donor regarding the frequency of squeeze. A completion indicator 546 appears when the blood collection completion has been completed.

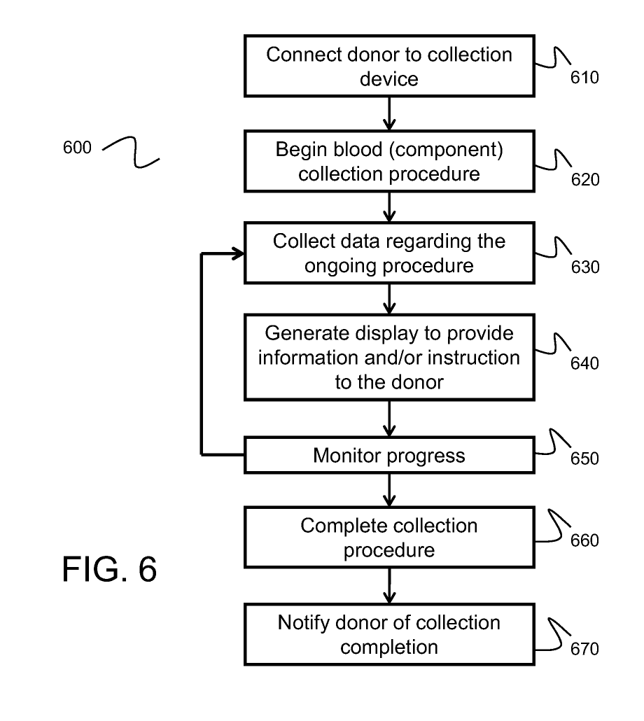

FIG. 6 is a flow diagram representative of example machine readable instructions that can be executed to implement the example systems and/or interfaces 100, 110, 120, 200, 210, 220, 300, 400-403, and/or 540-546 of FIGS. 1-5 and/or portions of one or more of those systems. The example processes of FIG. 6 can be performed using a processor, a controller and/or any other suitable processing device. For example, the example processes of FIG. 6 can be implemented using coded instructions (e.g., computer readable instructions) stored on a tangible computer readable medium such as a flash memory, a read-only memory (ROM), and/or a random-access memory (RAM). As used herein, the term tangible computer readable medium is expressly defined to include any type of computer readable storage and to exclude propagating signals. Additionally or alternatively, the example processes of FIG. 6 can be implemented using coded instructions (e.g., computer readable instructions) stored on a non-transitory computer readable medium such as a flash memory, a read-only memory (ROM), a random-access memory (RAM), a cache, or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

Alternatively, some or all of the example processes of FIG. 6 can be implemented using any combination(s) of application specific integrated circuit(s) (ASIC(s)), programmable logic device(s) (PLD(s)), field programmable logic device(s) (FPLD(s)), discrete logic, hardware, firmware, etc. Also, some or all of the example processes of FIG. 6 can be implemented manually or as any combination(s) of any of the foregoing techniques, for example, any combination of firmware, software, discrete logic and/or hardware. Further, although the example processes of FIG. 6 are described with reference to the flow diagrams of FIG. 6, other methods of implementing the processes of FIG. 6 can be employed. For example, the order of execution of the blocks can be changed, and/or some of the blocks described can be changed, eliminated, sub-divided, or combined. Additionally, any or all of the example processes of FIG. 6 can be performed sequentially and/or in parallel by, for example, separate processing threads, processors, devices, discrete logic, circuits, etc.

FIG. 6 depicts a flow diagram for an example method 600 for display of information and instructions to a donor. At block 610, a donor is connected to a collection device (e.g., an apheresis device, plasmapheresis device, etc.). For example, one or more needles and associated tubing is used to connect the donor to the device. The device is configured to collect all or a component of the donor's blood.

At block 620, the collection procedure begins for the donor using the device. For example, a plasma collection procedure begins with respect to the connected donor. At block 630, data related to the collection procedure is gathered. For example, data including donor blood flow, blood (component) volume collected, etc., is collected from one or more monitors and/or sensors in the collection device.

At block 640, a display output to provide information and/or instruction to the donor. For example, based on donor blood flow and volume collected in comparison to a target collection volume information regarding blood flow, volume collected versus target collection volume, etc., can be provided for display to the donor. In certain examples, the donor's target collection volume is provided on the donor display. The target collection volume corresponds to a value programmed into the blood collection device by an operator and/or automated program, for example. The value can be specific to that donor. The value can be shown on the donor display as part of a progress indicator and can also assist in verification of the target collection volume value by the donor, for example. Alternatively or in addition, instructions to help the donor increase or decrease his/her blood flow rate (e.g., squeeze harder, squeeze softer, squeeze more frequently, squeeze less frequently, etc.) can be generated based on the blood flow and volume data and provided via display to the donor, for example.

At block 650, donor collection progress is monitored. For example, data continues to be gathered to update the collection progress and update instructions to the donor as in blocks 630 and 640. At block 660, the collection procedure is completed. For example, a target donor plasma and/or other component volume is collected. At block 670, the donor is notified of procedure completion. For example, an audio and/or visual indicator can be provided to notify the door of the completion of the collection procedure.

Certain examples can be used in conjunction with one or more blood collection and/or processing devices and can be incorporated into a network of data communication and information exchange between a blood center, blood component collection instruments, and the like. For example, certain examples provide systems, apparatus, and/or methods for collecting, using, and storing information in a biological fluid collection and/or processing facility. Certain examples can be incorporated into an existing facility's system via an upgrade to existing hardware and software. Certain examples provide a data connection between laboratory instruments, including, but not limited to, existing blood and blood component collection instruments, such as the Autopheresis-C, ALYX, and/or AMICUS instruments which are supplied by Fenwal, Inc., such as those systems described in PCT Publication No. WO 01/17584, U.S. Pat. Nos. 5,581,687 and 5,956,023, and U.S. Ser. No. 09/037,356, which are incorporated by reference herein, and the collection facility's management information system which lends itself to automated tracing and/or tracking of donors and biological fluids data logging. Traceability can be provided via integration of donor, operator, soft goods, and instrument data. In certain examples, event reporting can be automated for regulatory compliance.

In certain examples, the instruments, laboratory equipment, as well as data input devices are connected to an Ethernet and/or other network along with other data processing applications. Certain examples are also suited for connecting legacy instruments that automatically transmit or can be configured to periodically transmit data via a serial or parallel interface and protocol converters. A computer acting as a server/gateway runs applications to receive the transmitted data and route them to database and hypertext markup language (HTML) applications. Each data packet bears a unique identifier which identifies the source of the data.

In certain examples, users can perform data query and reporting on a local area network, through a wide area network, over the Internet, or a combination of two or more of these, using a standard browser application interface. Real-time viewing and updating of device operation can be configured for any number of devices on the browser. In addition, the server also presents abbreviated data to a wireless personal digital assistant (PDA) also running a standard application browser interface for portable information and viewing and alarm and event notification. The PDAs are also used for data input (through a keypad touch screen, scanning, or other entering method—all used interchangeably herein) in association with an apparatus operation. Thus, certain examples include an open standard architecture in a heterogeneous apparatus environment with real-time update and access of data, and portable data viewing, reporting, notification, and inputting.

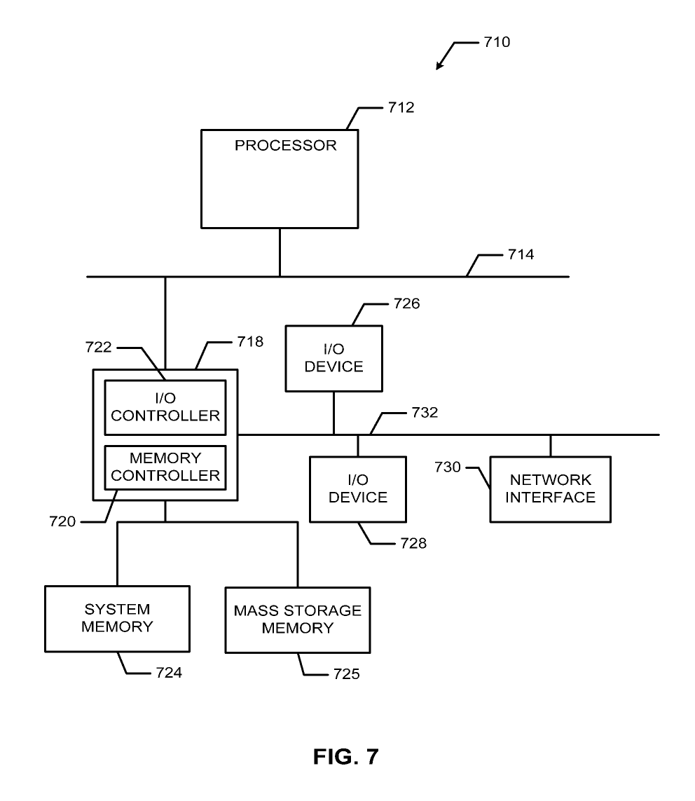

FIG. 7 is a block diagram of an example processor system 710 that can be used to implement systems, apparatus, and methods described herein. As shown in FIG. 7, the processor system 710 includes a processor 712 that is coupled to an interconnection bus 714. The processor 712 can be any suitable processor, processing unit, or microprocessor, for example. Although not shown in FIG. 7, the system 710 can be a multi-processor system and, thus, can include one or more additional processors that are identical or similar to the processor 712 and that are communicatively coupled to the interconnection bus 714.

The processor 712 of FIG. 7 is coupled to a chipset 718, which includes a memory controller 720 and an input/output (“I/O”) controller 722. As is well known, a chipset typically provides I/O and memory management functions as well as a plurality of general purpose and/or special purpose registers, timers, etc. that are accessible or used by one or more processors coupled to the chipset 718. The memory controller 720 performs functions that enable the processor 712 (or processors if there are multiple processors) to access a system memory 724 and a mass storage memory 725.

The system memory 724 can include any desired type of volatile and/or non-volatile memory such as, for example, static random access memory (SRAM), dynamic random access memory (DRAM), flash memory, read-only memory (ROM), etc. The mass storage memory 725 can include any desired type of mass storage device including hard disk drives, optical drives, tape storage devices, etc.

The I/O controller 722 performs functions that enable the processor 712 to communicate with peripheral input/output (“I/O”) devices 726 and 728 and a network interface 730 via an I/O bus 732. The I/O devices 726 and 728 can be any desired type of I/O device such as, for example, a keyboard, a video display or monitor, a mouse, etc. The network interface 730 can be, for example, an Ethernet device, an asynchronous transfer mode (“ATM”) device, an 802.11 device, a DSL modem, a cable modem, a cellular modem, etc., that enables the processor system 710 to communicate with another processor system.

While the memory controller 720 and the I/O controller 722 are depicted in FIG. 7 as separate blocks within the chipset 718, the functions performed by these blocks can be integrated within a single semiconductor circuit or can be implemented using two or more separate integrated circuits.

Certain embodiments contemplate methods, systems and computer program products on any machine-readable media to implement functionality described above. Certain embodiments can be implemented using an existing computer processor, or by a special purpose computer processor incorporated for this or another purpose or by a hardwired and/or firmware system, for example.

Some or all of the system, apparatus, and/or article of manufacture components described above, or parts thereof, can be implemented using instructions, code, and/or other software and/or firmware, etc. stored on a machine accessible or readable medium and executable by, for example, a processor system (e.g., the example processor system 710 of FIG. 7). When any of the appended claims are read to cover a purely software and/or firmware implementation, at least one of the components is hereby expressly defined to include a tangible medium such as a memory, DVD, CD, Blu-ray disc, etc. storing the software and/or firmware.

Thus, certain examples provide graphical display of information and instructions to a donor connected to a blood collection device for a blood collection procedure. Certain examples display graphical instructions to a donor regarding a desired flow rate and fist squeeze intensity to improve blood (component) collection and safeguard the donor.

While particular embodiments of the invention have been shown and described, it will be obvious to those skilled in the art that changes and modifications may be made therein without departing from the invention in its broader aspects.

It will be appreciated by persons skilled in the art that numerous variations and/or modifications may be made to the invention as shown in the specific embodiments and/or aspects without departing from the spirit or scope of the invention as broadly described. The present embodiments and aspects are, therefore, to be considered in all respects as illustrative and not restrictive. Several embodiments are described above with reference to the drawings. These drawings illustrate certain details of specific embodiments that implement the systems and methods and programs of the present invention. However, describing the invention with drawings should not be construed as imposing on the invention any limitations associated with features shown in the drawings. The present invention contemplates methods, systems and program products on any electronic device and/or machine-readable media suitable for accomplishing its operations. Certain embodiments of the present invention may be implemented using an existing computer processor and/or by a special purpose computer processor incorporated for this or another purpose or by a hardwired system, for example.

Embodiments within the scope of the present invention include program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

1. A blood collection system comprising:
an operator user interface to allow an operator to configure the system for a blood collection procedure from a donor;
a donor display, separate from the operator user interface, arranged in the system to be within view of the donor who is to donate via the system, the donor display to display information and instruction to the donor, the information and instruction comprising a first indicator regarding a progress of the blood collection procedure for the donor and a second indicator comprising a visual prompt for donor interaction in the blood collection procedure, the visual prompt dynamically generated based on obtained sensor feedback indicative of donor blood flow; and
a processor and a memory, the processor to execute instructions stored in the memory to process input from and provide output to the operator user interface and the donor display.

2. The system of claim 1, wherein the second indicator comprises a graphical representation of a fist.

3. The system of claim 2, wherein the second indicator comprises a graphical representation of a fist plus one or more bars to indicate at least one of a squeeze intensity and a squeeze frequency.

4. The system of claim 1, wherein the second indicator flashes at a variable rate based on a desired donor squeeze frequency.

5. The system of claim 1, wherein the second indicator changes color according to a desired donor squeeze intensity.

6. The system of claim 1, wherein a discrete completion symbol is displayed upon completion of blood collection procedure.

7. The system of claim 1, wherein the blood collection procedure comprises a plasmapheresis blood component collection procedure.

8. The system of claim 1, wherein the information and instruction are derived from collected sensor data regarding donor vein and blood pressure feedback and collected volume at the system.

9. The system of claim 1, wherein a target collection volume for the donor is provided on the donor display.

10. The system of claim 9, wherein the target collection volume corresponds to a value programmed into the blood collection system by the operator for the donor.

11. A tangible computer readable medium including executable program instructions which, when executed by a processor, cause the processor to provide:
an operator user interface to allow an operator to configure a blood collection system for a blood collection procedure from a donor;
a donor display, separate from the operator user interface, arranged in the blood collection system to be within view of the donor who is to donate via the blood collection system, the donor display to display information and instruction to the donor, the information and instruction comprising a first indicator regarding a progress of the blood collection procedure for the donor and a second indicator comprising a visual prompt for donor interaction in the blood collection procedure, the visual prompt dynamically generated based on obtained sensor feedback indicative of donor blood flow.

12. The computer readable medium of claim 11, wherein the second indicator comprises a graphical representation of a fist.

13. The computer readable medium of claim 12, wherein the second indicator comprises a graphical representation of a fist plus one or more bars.

14. The computer readable medium of claim 11, wherein the second indicator flashes according to a desired donor squeeze frequency.

15. The computer readable medium of claim 11, wherein the second indicator changes color according to a desired donor squeeze intensity.

16. The computer readable medium of claim 11, wherein a discrete completion symbol is displayed upon completion of blood collection procedure.

17. The computer readable medium of claim 11, wherein the blood collection procedure comprises a plasmapheresis blood component collection procedure.

18. The computer readable medium of claim 11, wherein the information and instruction are derived from collected sensor data regarding donor vein and blood pressure feedback and collected volume at the blood collection system.

19. A method for computer-assisted blood collection from a donor, the method comprising:
adaptively processing measured data regarding donor blood flow and collected blood component volume from an ongoing blood collection procedure at a blood collection device;
determining at least one of a desired donor hand squeeze intensity and a desired donor hand squeeze frequency based on the processed data;
generating a graphical indicator based on the at least one of the desired donor hand squeeze intensity and the desired donor hand squeeze frequency to instruct the donor regarding the at least one of donor hand squeeze intensity and donor hand squeeze frequency to facilitate the blood collection procedure; and
displaying the graphical indicator to the donor via a donor display arranged to be viewable by the donor during the ongoing blood collection procedure.

20. The method of claim 19, wherein the graphical indicator animates to instruct the donor regarding at least one of the desired donor squeeze frequency and the desired donor squeeze intensity.

21. The method of claim 19, wherein the graphical indicator comprises a graphical representation of a fist displayed alone or in conjunction with one or more secondary graphical indicators.

22. The method of claim 19, wherein the measured data includes data regarding donor vein and blood pressure feedback collected from the donor by one or more sensors.

23. The method of claim 19, further comprising displaying a target collection volume for the donor on the donor display.

24. The method of claim 19, wherein the target collection volume corresponds to a value programmed into the blood collection system by the operator for the donor.
